(19) United States
(12) Patent Application Publication
(10) Pub. No.: US 2022/0226815 A1
(43) Pub. Date: Jul. 21, 2022

(54) METHOD AND DEVICE FOR PARALLEL SINGLE-CELL PROCESSING

(71) Applicant: THE RESEARCH FOUNDATION FOR THE STATE UNIVERSITY OF NEW YORK, Albany, NY (US)

(72) Inventors: Eric BROUZES, Port Jefferson, NY (US); Jin Zhu YU, Flushing, NY (US); Anu Priya Bharathi RAJAN, Stony Brook, NY (US); Jingfang JU, East Setauket, NY (US); Gabor BALAZSI, East Setauket, NY (US)

(73) Assignee: THE RESEARCH FOUNDATION FOR THE STATE UNIVERSITY OF NEW YORK, Albany, NY (US)

(21) Appl. No.: 17/609,485

(22) PCT Filed: May 8, 2020
(86) PCT No.: PCT/US2020/032183

§ 371 Date: Nov. 8, 2021

Related U.S. Application Data
(60) Provisional application No. 62/845,494, filed on May 9, 2019.

(51) Int. Cl. B01L 3/00; C12Q 1/686

(52) U.S. Cl. CPC B01L 3/50851; C12Q 1/686; B01L 2400/049; B01L 2300/0681; B01L 2200/12; B01L 2300/021; B01L 2300/0829; B01L 2300/161

(57) ABSTRACT

The present disclosure is directed to a microwell array comprising a plurality of wells of micro-size dimensions created on porous materials. The device can be used in various cell and tissue analytical activities, and can be formed using an etching, laminating or imprinting processes.

### CROSS REFERENCE TO RELATED APPLICATION

This application claims the benefit of priority from U.S. Provisional Application No. 62/845,494, filed May 9, 2019, the entire contents of which are incorporated herein by reference.

### GOVERNMENT SPONSORSHIP

This invention was made with government support under HL 127522 awarded by the National Institutes of Health. The government has certain rights in the invention.

### BACKGROUND OF THE DISCLOSURE

Single-cell genomics is a set of methods that seek to obtain genomic information (copy number variant profile, genomic DNA, transcriptome, epigenetic state, etc.) of single-cells using quantitative polymerase chain reaction (PCR) or sequencing. These methods are used to extract genomic information from individual cells out of a complex mixture of cells, particularly in neurology, cell biology, immunology and cancer research.

The impact of single-cell genomics has been recognized by the community was named Method of the Year 2013 by Nature magazine. The applications of single-cell genomics are mostly concentrated on the analysis of very rare cells such as unculturable bacteria, embryos and circulating tumor cells, and on the analysis of heterogeneous tissues such as tumor.

Despite its potential, single-cell genomics is hindered by a laborious workflow that has historically been manual and low throughput. In addition, the required number of cells to analyze can also be difficult to attain with existing methods. One of the key drivers of the growth of the single-cell genomics market is the development of innovative platforms to streamline the sample preparation of single-cells at high throughput.

Presently, obtaining genomic information from single-cells entails 3 steps: 1—separate/capture single-cells into isolated sub-volumes, 2—cell processing/sample preparation, and 3—genomic analysis. The workflow is labor-intensive requiring a wide range of expertise, and it remains challenging, low throughput and mostly manual.

Current methods are based on: 1—arrays of on-chip valves which limits the number of cells that can be processed (up to 96 cells); 2—microfluidic droplets that rely on complicated actuation and manipulations, and that cannot simply address applications such as single-cell quantitative (q)PCR or Reverse Transcription (RT)-PCR; or 3—robotic based system that do not solve the throughput and simplicity issues. On a technical level, the main limitations of those methods are the limited number of cells analyzed and the limited number of times the systems can perform washes. As a result, workflows that can be used on those platforms are limited to very few steps (1 or 2), which excludes applications such as single-cell epigenomics.

What is desired is a relatively simple and effective method to perform molecular reactions at single-cell resolution. In contrast to existing methods, the approach herein 1) is comparatively simple to implement, 2) is directly compatible with most workflows for sample preparation, thus 3) enables new analyses at single-cell resolution such as epigenetics; and 4) permits the analysis of a very large number of single-cells at once. In summary, there is an unmet need for a simple to use platform that integrates cell isolation, sample preparation for a large number of single-cells. Additional unmet needs include solutions for 1) massively parallel single-cell qRT-PCR, and 2) single-cell epigenomics.

The disclosed methods and devices are configured to undertake both cell isolation and sample preparation; the disclosure will allow multi-step processing of massive numbers of single cells for single-cell genomics (100,000's). The present disclosure will also enable single-cell qRT-PCR and digital PCR.

Embodiments of the present disclosure provide devices and methods that address the above needs.

### SUMMARY OF THE DISCLOSURE

The present disclosure is directed to a microwell array comprising a plurality of wells etched into a porous glass. The array can be used in various cell analysis activities, and can be formed using an etching process.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will be better understood by reference to the following drawings, which are provided as illustrative of certain embodiments of the subject application, and not meant to limit the scope of the present disclosure.

FIGS. 1A-1E are illustrations of arrays of microwells and a solution and a single-cell solution.

FIGS. 2A-2E illustrate schematics of components of the present disclosure, and operations that can be performed.

FIGS. 3A-3C illustrate a platform prototype.

FIGS. 4A-4C are illustrations of a method of using an array of the present disclosure.

FIG. 5 is an illustration of a workflow demonstrating the seal-unseal cycles with a single-cell array device.

FIGS. 6A-6E are photographs illustrating efficient wash seal-unseal and buffer exchange cycles in single-cell devices without single-cell displacement. FIG. 6A is stitched Fluorescent micrographs of microwell array with WGA-Texas Red pre-stained K562 cells; FIG. 6B is fluorescent micrographs of microwell array after oil sealing; FIG. 6C is fluorescent micrographs of microwell array after addition of SYTO 24 green dye; FIG. 6D fluorescent micrographs of microwell array after second oil sealing; FIG. 6E is a magnified section of the stitched fluorescent micrographs.

FIGS. 7A-7D are images of fluorescent micrographs of the microwell array with WGA-Texas Red pre-stained K562 cells. FIG. 7B is a fluorescent micrograph of the microwell array after the addition of SYTO 24 green fluorescent dye; FIG. 7C is a merged image of FIGS. 7A and 7B; and FIG. 7D is a magnified view of a section of the stitched micrograph.

FIGS. 8A-8C illustrate a single-cell RT-qPCR workflow. FIG. 8D is an illustration of direct workflow entailing nuclei transfer followed by combinatorial printing of two barcodes. FIGS. 8E-8H illustrate a barcode synthesis process. FIG. 8I illustrates tissue processing steps using the device of the disclosure.

FIGS. 9A-9C are photographs of a microwell device and accompanying vacuum unit.

FIGS. 10A-10D are fluorescent micrographs demonstrating the wash and seal-unseal capabilities of the microwell array device of the disclosure.

FIG. 11 is an illustration of a number of steps for imaging cells taken from the liver of a mouse.

FIGS. 12A-12F are micrographs of cell samples under various conditions.

FIGS. 13A-13C are fluorescent micrographs of tissue samples.

FIG. 14, is an image of a microwell array obtained with a stereomicroscope.

FIG. 15 is an illustration of a design of one microwell designed with a Computer Assisted Design (CAD) software.

FIG. 16 is an illustration of a portion of a microwell array designed with a Computer Assisted Design software.

FIG. 17 is a CAD design used to create a photomask that can be used to form a number of microwell arrays.

FIG. 18 is an illustration of an example of the Stacking Order of imprinting elements. The pattern of the 5:1 PDMS mold is the negative of the array of microwells.

FIG. 19 is an image of a microwell array created in thermoplastic.

FIGS. 20A-20C are illustrations of the method steps of an example of imprinting elements to create an array of microwells onto a thin membrane of 0.2 micron pore size.

FIG. 21 is an image of a Single Sided Through-Hole Imprinted 30 um Thick Polystyrene Film Bonded to Cellulose Acetate 0.2 um Pore Syringe Membrane

FIGS. 22A and 22B are images of Single Sided Polystyrene Filters Bonded to 0.2 um Cellulose Acetate Syringe Filter Membranes.

FIGS. 23A and 23B are images of Single Sided Polystyrene microwell array Bonded to 0.2 um PTFE Syringe Filter Membranes.

FIGS. 24A and 24B are images of a single side PS surface.

FIG. 25 is an image of a PS array of microwells fabricated onto a cellulose acetate 0.2 micron pore membrane.

FIG. 26 is an image of a PS array of microwells fabricated onto a PTFE 0.2 micron pore membrane.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element, and a second element, are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the term “microwell,” generally refers to a well with a volume of less than about 1 mL. Microwells may be made in various volumes, larger or smaller than about 1 mL, depending on the application. In other embodiments, the term “microwell” may refer to a topological feature such as a well, a pit, a depression and similar, in which at least one of the linear dimensions is greater than about 1 micron but smaller than about 1 mm. However, in some embodiments, wells with linear dimensions greater than 1 mm may be also referred to as microwells.

The present disclosure includes devices and methods for cell capture and/or analysis. These devices have numerous applications in such diverse fields as developmental biology, immunology, neuroscience, cancer research, basic cell biology, stem cell biology and microbiology.

Many current approaches have reduced the molecular biology of the workflow to a single step because the microfluidic formats used do not allow for multiple-step workflows with washing steps. This results in an unacceptable compromise that directly affects the quality of the data generated and precludes several applications altogether. In addition, current technologies involve complex and expensive operations that rely either on active encapsulation or on-chip microvalves.

One difference realized by the disclosed microwell array is the method to fabricate the bottom plate and its shape, which are discussed below. Typical methods and devices cannot lyse and retain molecules of interest. Also, typical methods and devices cannot unseal by aspirating a layer of immiscible oil.

The disclosed microwell array includes a frit filter made of a glass (or other suitable material). Crosstalk through the filter base to other microwells is reduced or prevented by control of surface properties.

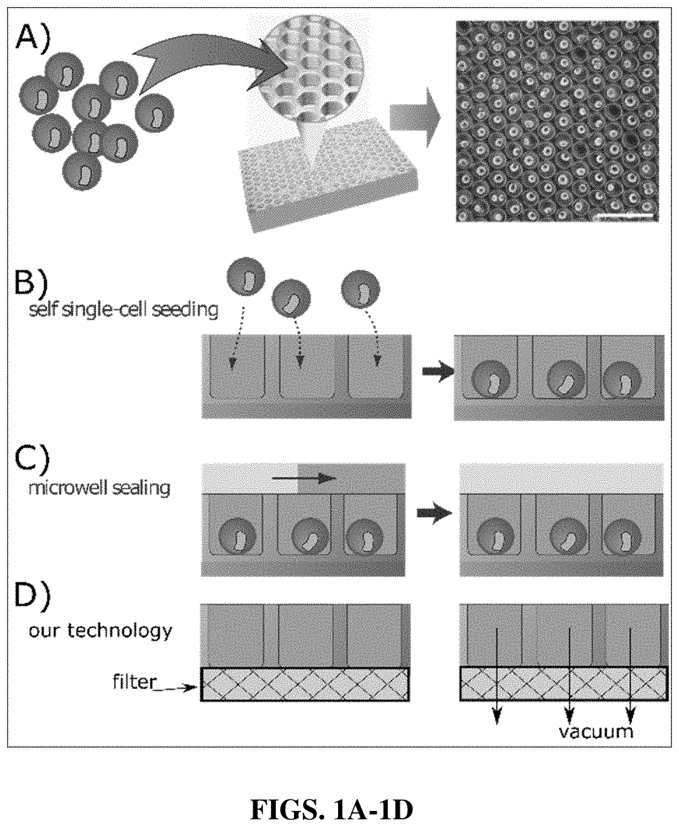

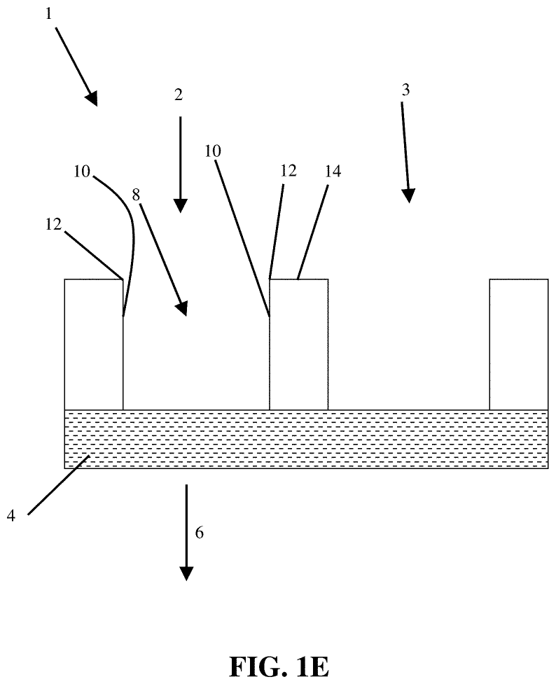

The present application is based on a modified array of microwells where microwells are fabricated on top of a glass filter (FIGS. 1A-1D). This allows the use of vacuum actuation to flow liquids through the microwells. FIGS. 1A-1D illustrate an array of microwells. FIG. 1A illustrates arrays of microwells enable simple single-cell partitioning via self single-cell seeding (FIG. 1B). FIG. 1C illustrates the microwell being sealed into independent reactors. FIG. 1D illustrates the exchange of buffer and unsealing microwells due to a bottom plate made of a frit filter. More details regarding this disclosure are included below.

Arrays of microwells can be used to array single-cells. Microwells can be isolated from each other and thus used to easily create a large number (100,000's) of independent reactors. However, unsealing those reactors to perform further operations and washing steps have previously remained elusive. The present disclosure addresses the previous issues.

An annotated view of a microwell array, shown in FIG. 1D, is illustrated in FIG. 1E. FIG. 1E is a cross sectional view of a microwell array, with the cross section passing through two wells of the microwell array. The wells can be cylindrically shaped, can include shapes with one or more sides, or any other suitable shape.

In FIG. 1E, a microwell array 1 is shown, with a well 2. Adjacent the well 2 in the microwell array is an adjacent well 3. Although only two wells of the microwell array 1 are shown in FIG. 1E, the microwell array 1 can include many wells in many different locations. The wells of the microwell array 1 are etched into a porous substrate, such as glass, or other suitable material.

The microwell array 1 includes a bottom surface 4. The wells of the microwell array 1 are deposited on an upper surface of the bottom surface 4. In certain embodiments, the bottom surface 4 is constructed of porous glass, though other suitable materials may be used. The bottom surface 4 is configured to receive and transmit a vacuum pressure (shown as moving in the direction of arrow 6) through the bottom surface 4 to an interior volume 8 of the well 2. The interior volume 8 is comprised of a side wall 10 and the bottom surface 4. The bottom surface 4 is either hydrophobic or hydrophilic. In certain embodiments, the bottom surface 4 has a pore size of about 4 microns to about 8 microns, about 2 microns to about 2.5 microns, about 0.9 microns to about 1.4 microns, or about 10 microns to about 20 microns.

The side wall 10 is hydrophilic. The side wall 10 may be porous or non-porous. In certain embodiments, the side wall 10 is constructed of a deposited metal, such as by electrodeposition and/or electroplating of a conductive metal, such as copper and/or nickel. The side wall 10 includes an upper edge 12. In certain embodiments, the side wall 10 has a curved surface, that is, when the well 2 is cylindrical in shape, In certain embodiments, the side wall 10 has a flat surface, that is, when the well 2 has a square or polygonal shape. The upper edge 12 can be a relatively thin line at or near the vertical top of the side wall 10 or can be a region at or near the vertical top of the side wall 10. In certain embodiments, the upper edge 12 of the side wall 10 is hydrophobic or fluorophilic. Also, the upper edge 12 can extend at least partially along an upper surface 14. The upper surface 14 is a space between the upper edge 12 of adjacent wells (well 2 and adjacent well 3). A space between the upper edge 12 of two adjacent wells 2 and 3 forms an upper surface, and in certain embodiments, the upper surface is hydrophobic.

As used herein “hydrophilic” refers to molecules and/or components of molecules having at least one hydrophilic group, and “hydrophobic” refers to molecules and/or components of molecules having at least one hydrophobic group. Hydrophilic molecules or components thereof tend to have ionic and/or polar groups, and hydrophobic molecules or components thereof tend to have nonionic and/or nonpolar groups. Hydrophilic molecules or components thereof tend to participate in stabilizing interactions with an aqueous solution, including hydrogen bonding and dipole-dipole interactions. Hydrophobic molecules or components tend not to participate in stabilizing interactions with an aqueous solution and, thus often cluster together in an aqueous solution to achieve a more stable thermodynamic state.

As used herein “fluorophilic” refers to molecules and/or components of molecules having at least one fluorophilic group. A fluorophilic group is one that is capable of participating in stabilizing interactions with a fluorous phase. Fluorophilic groups useful in block copolymers of the present disclosure include, but are not limited to, fluorocarbon groups, perfluorinated groups and semifluorinated groups.

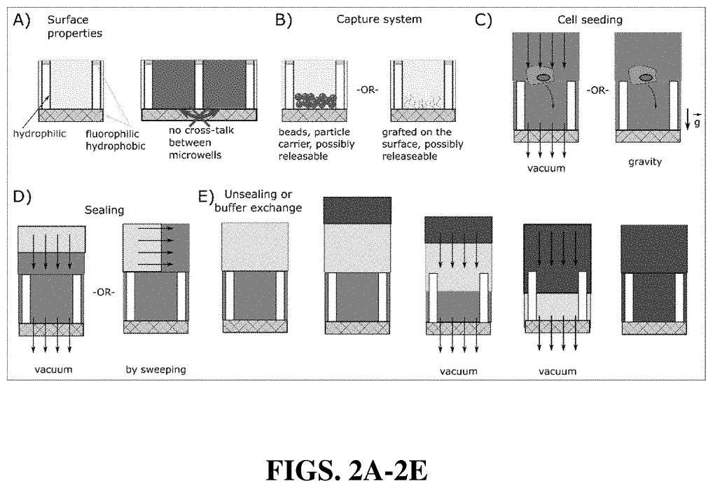

As further described with respect to FIG. 2B, in certain embodiments, multiple beads are disposed in each of the wells. The beads are configured to substantially fill the pores in the bottom surface 4.

It is noted that the various surfaces of the microwell array 1 described herein may have different properties while being constructed of the same material. The properties of the various surfaces of the microwell array 1 are a result of the sequential etching process further described herein. For example, the sidewall 10 may be hydrophilic and non-porous while the bottom surface 4 is hydrophobic and porous. The sidewall 10 may be hydrophilic and porous while the bottom surface 4 is hydrophobic and porous. The sidewall 10 may be hydrophilic and non-porous while the bottom surface 4 is hydrophilic and porous. The sidewall 10 may be hydrophilic and porous while the bottom surface 4 is hydrophilic and porous. The upper edge 12 of the side wall 10 may be hydrophobic or fluorophilic when combined with any of the above embodiments.

In certain embodiments, the microwell array 1 comprises a plurality of first barcodes and a plurality of second barcodes. The plurality of first barcodes are printed on at least one of the side wall 10 and the bottom surface 4. The plurality of second barcodes are printed on at least one of the side wall 10 and the bottom surface 4. The first and second barcodes are printed in the manner further described herein. In certain embodiments, the plurality of first barcodes are printed on a first particle. The interior volume 8 of the well 2 comprises the first particle. In certain embodiments, the plurality of first barcodes are printed on a second particle and the interior volume 8 of the well 2 comprises the second particle. The purpose of the first and second barcodes is to be able to identify the location of the well 2 or the particle that is being investigated.

The first and/or second barcodes can be attached to the first and second particles, in any suitable way, such as by printing, as well as by grafting and/or by synthesizing the barcodes directly onto the surface of the particles.

Two components of the present disclosure include: 1) an array of microwells sitting on top of a glass filter where the top surface of the array and the bulk of the glass filter are substantially hydrophobic or fluorophilic and where the microwells inner surfaces, which can include the top surface of the glass filter, are hydrophilic (as seen in FIG. 2A); and 2) a capture system that includes oligonucleotides, aptamers, or antibodies that can be grafted onto beads or particles or directly grafted at the surfaces of the microwells (as seen in FIG. 2B). The surface treatment prevents aqueous phase to bridge microwells and keep them independent (as seen in FIG. 2A). Said another way, this treatment and design allows for a liquid to move vertically downward upon application of a vacuum through the bottom surface, not horizontally between wells themselves.

The operation of the present disclosure can be seen in FIGS. 2A-2E, such as in FIG. 2C single-cell seeding with/out vacuum is performed, then microwell sealing with/out vacuum is performed as seen in FIG. 2D, and then unsealing and buffer exchange without displacing single-cells is performed, as seen in FIG. 2E.

Again, FIG. 2A illustrates surface properties to prevent crosstalk between microwells. FIG. 2B illustrates a capture system to retain molecules of interest. FIG. 2C illustrates single-cell seeding. FIG. 2D illustrates a sealing operation with immiscible oil (yellow). FIG. 2E illustrates unsealing and buffer exchange (green buffer), operations.

Further, as shown in FIG. 2A, the bulk of the filter is hydrophobic to prevent crosstalk between the microwells through the filter, and the inner surfaces of the microwells are hydrophilic to retain aqueous solutions. The hydrophobicity can be obtained by silane treatment with fluorinated molecules or other hydrophobic molecules. This treatment can be performed by treating the entire filter into a silanisation solution.

The inner walls of the microwells are hydrophilic to support the loading of aqueous buffers. The inner walls or surfaces can include the top surface of the filter. If a global silanisation reaction is used to make the filter hydrophobic, the top surface can be reverted to being hydrophilic by etching. Alternatively, the top surface could be protected during the hydrophobic silanisation of the filter.

Further, the other inner surfaces of the microwells can be assured to be hydrophilic by oxidation or by surface treatment with functionalized PEG molecules and/or by treatment with APTES ((3-Aminopropyl)triethoxysilane). Depending on the applications, the top surface (upper edge) can be rendered hydrophobic with a silane treatment by contact printing to restrict the silane reaction to the very top surface of the array without affecting the inner surfaces of the microwells. The top surface could also be surface treated with silane PEG or fluorinated or hydrophobic molecules. The depth of the hydrophobicity of the upper edge can be relatively shallow. The sequential steps discussed in the preceding paragraphs allows for control of the properties of the different surfaces of the microwells.

The array of microwells in FIG. 2A can be fabricated with a photoresist, either liquid or dry. A dry photoresist (TMMF), can be laminated layer by layer on top of the glass filter. Photolithography is then used to pattern the photoresist and develop the unexposed structures of the film.

Alternatively, those microwell structures could be obtained by (1) direct etching of the glass filter using hydrogen fluoride (or hydrofluoric acid) in liquid or gas form. In this example, the etching is structured due to the fabrication of a chemical mask such as a layer of chromium that is itself patterned by photo-lithography using a photoresist;

(2) direct etching after reflowing the top surface of the glass filter so that the top and thin layer is made first as a solid glass but etching gives access to the porous structure of the filter. The rest of the procedure will be similar to (1).

(3) electrodeposition where the walls of the microwells are obtained by addition of metal via an electrochemical reaction. In this embodiment, the deposition can be performed after creating a mold on the glass filter.

(4) bonding of a flow-through array of microwells made of plastic (polystyrene, cyclic olefin co-polymer, etc.).

(5) without etching the top surface of the glass filter, thus keeping it fluorophilic. This manufacturing option has an advantage if there is a concern about cross-contamination between the wells through the filter itself. Under this option, at the bottom of each well can include a relatively thin layer of oil to substantially prevent or substantially reduce communication of fluid between the wells through the glass filter itself.

The porosity of the filter is used to retain cells, particles, nuclei, tissue bits or pieces to be processed. Filters could be 7176 sintered glass filter discs porosity D (about 10 microns to about 20 microns), E (about 4-about 8 microns), very fine (about 2-about 2.5 microns), and/or ultra fine (about 0.9-about 1.4 micron). Alternatively, the glass filter can be of a larger porosity than the entities it needs to retain if it is used in conjunction with beads of the appropriate size that will fill the pores and reduce the effective pore size of the system.

This inception can be used to reduce the overall resistance of the flow through the device while assuring the correct effective pore size. The filter could be made of any material that is compatible with the functional requirements, i.e. hydrophobic bulk and hydrophilic top surface.

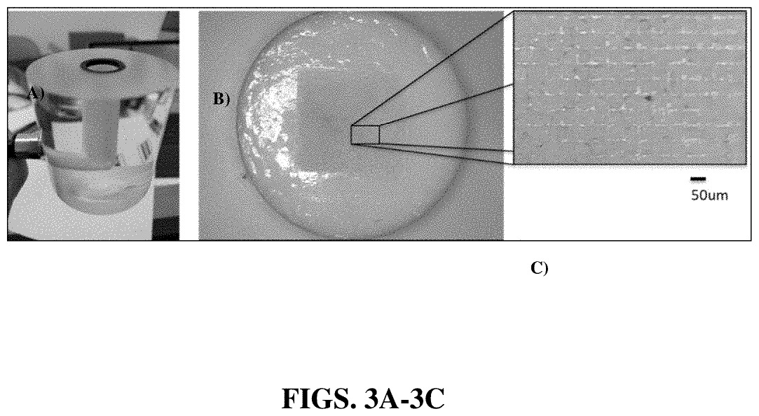

Photographs of an array of microwells is shown in FIGS. 3A-3C, which illustrate a platform prototype. FIG. 3A is a photograph of the vacuum holder of the array. FIG. 3B is a top view photograph of a glass filter with an array of microwells manufactured by optical lithography with TMMF photoresist, as discussed in the examples below. FIG. 3C is a magnified view of the microwells of FIG. 3B.

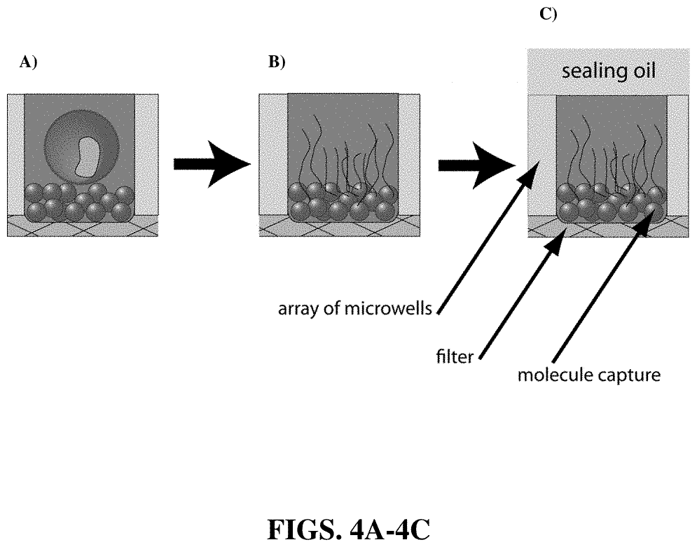

FIGS. 4A-4C are illustrations of a method of using a filter, such as the one photographed in FIGS. 3B and 3C. FIG. 4A illustrates the bottom of an array of microwells, with the bottom surface being a glass filter and a number of molecules within the well. FIG. 4B illustrates the capture system, with FIG. 4C illustrating the retention of the molecules of interest within the array of microwells, after application of a vacuum through the filter, during washing or unsealing of the microwell. As can be seen a sealing oil is placed over the microwell to aid in retention of the molecules in the array of microwells.

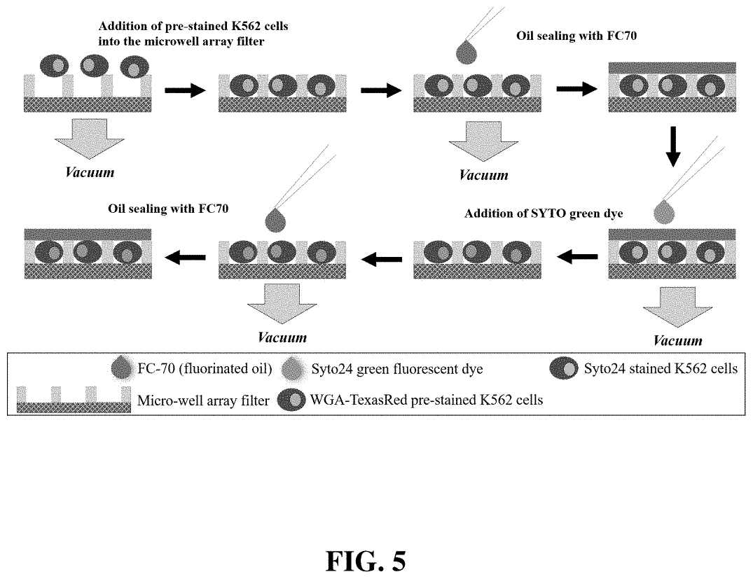

FIG. 5 is an illustration of an exemplary workflow demonstrating the seal-unseal cycles with a single-cell array device. Single-cells were seeded and diluted enough to form a specific pattern of filled and empty wells, with the first portion of FIG. 5 illustrating the addition of pre-stained K562 cells to the filter, application of a vacuum to the bottom surface of the filter, causing the three illustrated wells to receive the cells. A further vacuum can be applied, followed by application of a sealing oil to an upper surface of the filter.

The method of using the array device can include optional steps, which are illustrated in FIG. 5, including addition of a further dye to the cells (such as SYTO green dye) application of further sealing oils and application of the vacuum one or more times.

The methods and model of the present disclosure will be better understood by reference to the following Examples, which are provided as exemplary of the disclosure and not in any way limiting.

### Example 1

The device was manufactured using glass filters from Ace Glass as the base and fabricating the array of microwells using a dry film photoresist (TMMF) laminated on the filter base. Two devices were manufactured, one for single-cell applications and one for tissue analysis. A design constraint on the microwell size is the ability to perform single-cell applications. A design constraint for the tissue analysis is a microwell size for tissue analysis, being set to 70-100 μm, which allows barcode printing with a bioprinter.

One strategy to fabricate the single-cell device with the required properties includes the following workflow: grafting fluorophilic tri-chloro silane (TCS) based molecules onto the glass filter (other molecules could be used such as mono or di-chloro grafting moieties); etching the top of the glass filter to recover hydrophilicity of that face; laminating the dry photoresist; etching the dry photoresist to create microwells; activating the top surface of the device with oxygen plasma to make it reactive to tri chloro silane molecule; and grafting fluorophilic molecules to the top of the array by stamping the array onto a substrate coated with tri-chloro silane molecules.

One strategy to fabricate the tissue analysis device with the required properties includes the following workflow: dicing tissue sections (slices or chunks) into single cells or multiple adjacent cells; applying vacuum suction to absorb tissue into well by disintegrating tissue connectivity or break apart the one-piece tissue; preserving the location of each cell or multiple adjacent cells within the tissue; the bottom of the wells is a membrane that allow capture of the tissue parts, removal of buffer/reagent such as digestion enzyme after cells are captured in the well; and applying vacuum pressure to pull buffer (administered from the top) through membrane.

In this embodiment, the membranous bottom is a physical barrier that can retain cells after tissue dicing from the vacuum suction, and wells can be pre-treated or pre-loaded with labeling agents to retain only target of interest, such as a specific protein or nucleic acid, etc.

This capture system can be bead based, magnetic bead based, and/or grafted molecule, with the molecules that can be retained being mRNAs, proteins, lipids or any molecule that can be captured with the appropriate capture system. With the tissue capture system, diagnostic/histologic analysis of single or multiple adjacent cells within each well can reveal the heterogeneity of cells within the same piece of tissue.

The wells can be pre-loaded with known and specific barcodes to label nucleic acids present after dicing, these labels will allow an assignment of sequences to the initial well and hence link spatial and sequencing information. Barcode pre-loading can be performed by printing or deposition of primers covered with a responsive hydrogel, by synthesis in-situ on the membrane or on particles (and/or beads), or by deposition of barcode-harboring particles (and/or beads). If particles (and/or beads) are used with the capture system, they can be deposited by FACS machine or passively, and they could contain a specific fluorescent signature to decipher their code

Also demonstrated in this example is the ability to unseal and exchange buffer in a single operation without displacing single-cells, by performing the following steps: 1) K562 cells were loaded, pre-stained with a membrane marker and at sub-optimal density to create patterns of empty and filled microwells; 2) the microwells were sealed with a fluorinated oil (FC-70); 3) a nuclear staining solution was added (SYTO 24) on top of the oil layer; 4) a vacuum was applied to aspirate the oil layer (unseal) and exchange the solution. The operation proceeded successfully as proved by the nuclear staining of all the cells and the maintenance of cellular patterns within the array, as discussed below.

### Example 2

An experiment was devised to fully test the ability of the single-cell device to perform 1) wash and seal-unseal cycles, and (2) without displacing the single-cells.

This experiment included five steps, as follows, which are illustrated in FIG. 5, mentioned above:

Step 1 begins in the upper left portion of FIG. 5 and is: K562 stained with Wheat Germ Agglutinin labeled with Texas Red fluorophore were loaded to reveal their membrane and identify the presence of a cell in a microwell. The cells were seeded at intermediate concentrations to obtain random patterns of filled/empty wells at ratios around 70/30. The patterns generated were compared at each step of the experiment to check whether single-cells would move between wells during the different operations.

Step 2: the microwells were sealed with the FC70 fluorinated oil, which can be done by applying vacuum (low pressure) or a sweeping of the cell buffer with the oil.

Step 3: a solution of nuclear stain (Syto24, a green fluorescent dye) was deposited on top of the protective oil layer.

Step 4: vacuum (higher pressure as compared to Step 2) was applied to aspirate the oil layer and exchange the cell buffer with the solution containing the nuclear dye. An alternative to this Step 4 is applying a vacuum to remove the oil first before adding another aqueous solution to the cells, possibly under moderate vacuum to maintain cells at their location.

Step 5: microwells were sealed again and the cycle can start again.

An efficient buffer exchange would result in cells stained both red and green while maintaining the pattern created by the cell occupancy. Using the above steps, the following data were gathered.

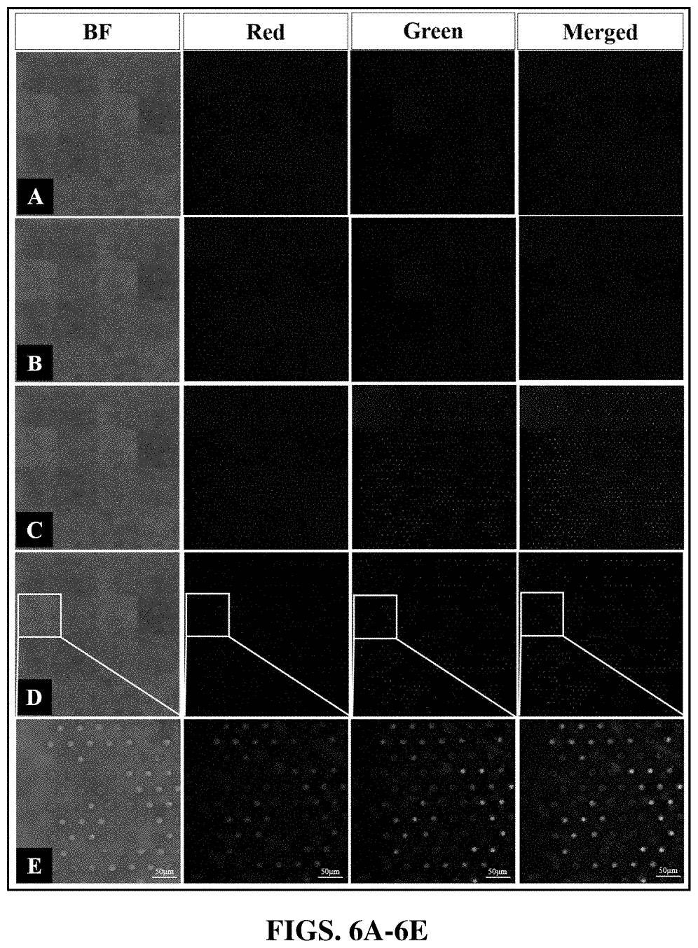

FIG. 6A shows the microwells occupied with WGA-Texas Red pre-stained K562 cells. It is observed that the cells occupy at least 70-75% of the microwells. Even though there are few cells that can be seen on the surface between two outside the wells, they get washed away when washed with PBS buffer and sealed with the oil (as seen in FIG. 6B). Sealing of the microwells with FC-70 fluorinated oil helped the transformation of an array of microwells to an array of independent reactor vessels. The addition of WGA-Texas Red X conjugate dye gave red fluorescence to the cells and since there is no other fluorescence at this point, the merged images show only red fluorescence. FIG. 6C shows the images that were taken after the addition of SYTO green fluorescence dye. Since the cells fluoresce for both red and green wavelengths, we can conclude that the cells nuclei took up the SYTO green dye indicating that the microwell array device enables efficient buffer exchange. From merging the images of the microwell array taken before and after the addition of SYTO dye and analyzing the pattern of cell occupancy in the images indicate that there is no displacement of cells during the buffer exchange.

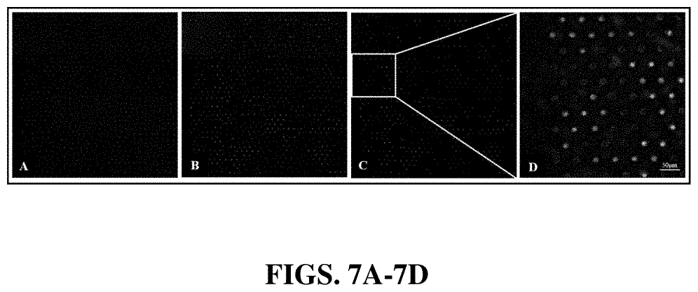

Also, by merging the images taken before and after the addition of green dye as shown in FIGS. 7A-7D it is shown that the cells were not displaced during the buffer exchange process. From this experiment it can be concluded that the oil-aspiration and buffer exchange can be done without displacing the cells in the microwell array.

### Example 3

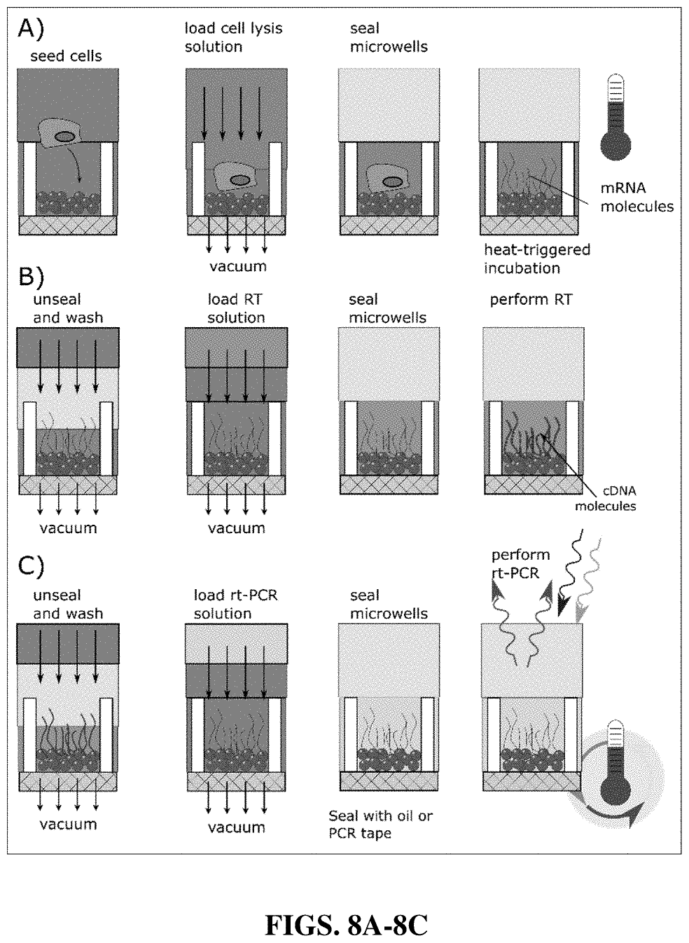

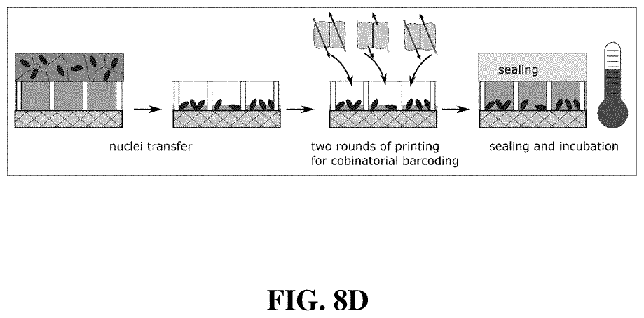

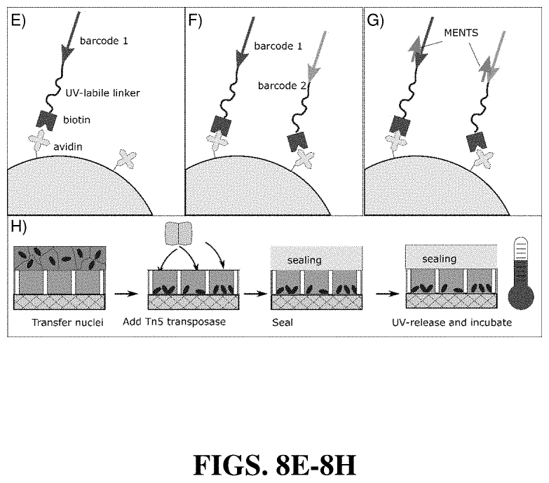

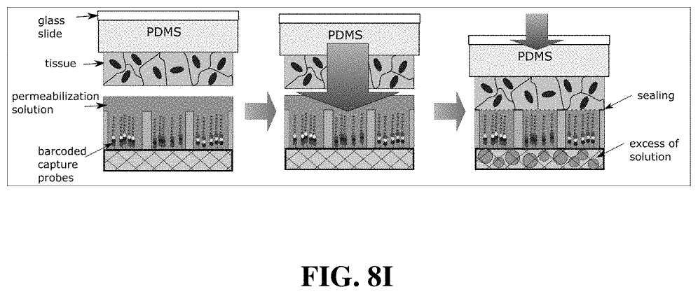

FIGS. 8A-8C illustrate a single-cell RT-qPCR workflow. Workflow for the massively parallel single-cell RT-qPCR application on the microwell of the present disclosure.

FIG. 8A illustrates cell seeding and lysis, moving from left to right in the figure. A single cell occupies each microwell that contains oligo-dT beads to capture mRNAs. The cell buffer is exchanged with a cell lysis solution that is activated with temperature after sealing of microwells with a layer of immiscible oil. The reaction is incubated to optimize the capture of mRNA molecules.

FIG. 8B illustrates how the microwells are unsealed and their content washed to remove any cell debris and DNA, moving from the left to the right in the figure. The washing solution is exchanged with a RT solution and the reaction is performed after sealing of the microwells with a layer of substantially immiscible oil.

FIG. 8C illustrates how the microwells are unsealed and their content washed, moving from left to right in the figure. That step could include RNAseH enzyme to remove the RNA molecules. The solution is then exchanged with the real-time PCR reagents. The real-time PCR is performed after sealing of the microwells with a layer of substantially immiscible oil and/or with PCR tape. The fluorescent signal from each microwell is recorded at the end of each temperature cycle.

Along with this step of performing PCR to the one or more cells, the disclosure includes, as an optional step, adding a barcode sequence to the one or more cells, and forming a sequencing library.

The term “barcode sequence” as used herein, refers to any ancillary nucleic acid sequence, which is pre-loaded or added after molecule capture, that is added to an insert nucleic acid sequence comprising at least one terminal overhang to which a barcode sequence may hybridize.

The term “library”, as used herein refers to a clone library, or alternatively, a library of genome-derived sequences carrying vector sequences. The library may also have sequences allowing amplification of the “library” by the PCR or other in vitro amplification methods well known to those skilled in the art. The library may also have sequences that are compatible with next-generation high throughput sequencers including but not limited to Illumina adapter pair sequences.

Barcodes can be pre-printed inside the microwells before use of the device to simplify the workflow for the end-user. The barcodes can be anchored to a surface for retention during nuclei transfer and washing steps. The barcodes can additionally be releasable on-demand to form active Tn5 transposase complexes.

Photocleavable linkers can be inserted into oligonucleotides (Integrated DNA Technologies). A UV-labile linker can be inserted in-between the 5′ end of the oligonucleotides and a biotin molecule (FIG. 8D). The oligonucleotides can be captured by avidin-functionalized 2 μm beads pre-loaded into the microwells. The first barcode can be printed at a non-saturating concentration. The unbound oligonucleotides will be washed away using vacuum actuation. The complementary 5′-phosphorylated MENTS, which is the same for all the wells, will be added to form the semi-duplex necessary for the Tn5 transposase. The hybridization will be conducted by a denaturation at 95° C. followed by a controlled cooling ramp using a thermocycler, which is based on the OpenPCR instrument (www.openpcr.org).

When exposed to UV light the linker is cleaved and the oligonucleotide with a 5′ phosphate will be released and ready to form a complex with the Tn5 transposase. The Tn5 transposase complexes are formed after nuclei transfer.

In anticipation for this scenario, tagmentation reactions where the Tn5 transposase is first pre-assembled (typical workflow) were compared to being directly mixed with genomic DNA and the barcodes. The reactions were performed on the same number of nuclei of a lung cancer cell line, HCC2450.

Similar Tn5 enzymatic activity was realized and assessed by enrichment qPCR and DNA fragment size distribution measured using the Bioanalyzer DNA High Sensitivity kit. This indicates that nuclei can be first transferred, the Tn5 transposase then loaded and the barcodes finally released to perform tagmentation.

These steps are illustrated in FIGS. 8E-8H. FIG. 8E illustrates a workflow with pre-printed barcodes entailing the pre-printing of barcodes that will be captured by avidin-functionalized microbeads. The biotin modification is added to the 3′ extremity of barcodes via a UV-labile linker. FIG. 8F illustrates that two barcodes are sequentially printed in each microwell to create a unique barcoding combination. FIG. 8G illustrates that the Mosaic End Non-Transferred Strand or MENTS (MENTS) is then added and hybridized. FIG. 8H illustrates how the nuclei are transferred into microwells and washed. The Tn5 transposase is added and the microwells sealed. The barcodes are released upon UV illumination and the tagmentation reaction is initiated at 37° C.

Further, to maintain high spatial resolution and limit lateral diffusion of mRNA molecules, a protocol to transfer mRNA molecules into underlying microwells was developed.

This protocol involves capturing mRNA molecules as close as possible to their location in the tissue. At small scale, the diffusion of mRNAs will blur their position and degrade the spatial resolution of the method. This effect can be limited by developing a two-prong approach that includes 1) using the closed microwells format and 2) limiting permeabilization to a single side of the tissue.

Under this approach, the tissue will be deposited on a glass slide coated with a layer of PDMS. PDMS is a silicon elastomer that will provide some flexibility when pressing the tissue onto the device. The thin layer of PDMS will allow imaging of the tissue again after its positioning onto the device to register its relative position onto the array of microwells. The device can be pre-loaded with a solution of pepsin following established protocols (FIG. 8I). Upon application of the tissue, the excess solution will go through the bottom filter. This approach will thus optimize the capture of mRNA molecules by providing a long incubation time without the limitation of molecules diffusing between capture spots.

As shown in FIG. 8I, tissue processing with the device is illustrated. The device is pre-loaded with a solution to permeabilize the tissue. The tissue mounted on a glass slide coated with a thin layer of PDMS is pressed gently against the device. Some pressure is maintained on the assembly (vacuum can be used in lieu of mechanical pressure) to create a seal between the microwell walls and the tissue so that mRNA molecules can only diffuse into the underlying microwell.

### Example 4

This example includes protocols for device fabrication, specifically, as one example, fabrication of the arrays of microwells using photolithography with a dry film photoresist.

As a first step, glass filters are subject to silanization and etching. In this example, 12 mm glass filters are first surface modified before using it as a substrate for making microwell array chip. For this purpose, the filters were first immersed in a 1% HEPTADECAFLUORO-1,1,2,2-TETRAHYDRODECYL)TRICHLOROSILANE in HFE7500 oil for an hour and then heated on a hot plate at a temperature above 100° C., to evaporate the oil. Next, the filters were etched on one of the surfaces of the filter using Armour etching paste for 5 min, followed by a washing to remove remaining paste and the glass that was etched.

The glass filters were then cleaned by immersing in deionized water (DI water) and then put under ultra-sonication for 20 min at 33° C. After ultra-sonication, the glass filters were rinsed with DI water and Isopropyl alcohol (IPA) before drying with N₂ gas. After drying, the filters were subject to a dehydration bake where they were baked at 200° C. for 10 min.

To make the disclosed device, TMMF S2045 can be used, which has a 45 μm thickness. The dry film photoresist contains 3 layers—a first Polyethylene terephthalate (PET) protective layer—a photoresist film—and a second PET protective layer. The PET layers protect the photoresist from dirt and the surroundings. So, before laminating, one of the PET layer is removed with help of the tweezers and then the photoresist is placed on top of the glass slide and placed between the rollers on the laminator (Prolam-Photo —Akiles ProLam Photo 6-Roller 13″ Pouch Photo Laminator; Roller speeds: 1/2/3/4/5/6/7/8/9 are respectively: 30/45/60/75/90/105/120/135/150 cm/minute). The roller temperature was set to 90° C. and the speed of the roller was set for 1 (slowest speed, about 30 cm/minute).

A bake was then performed at 100° C. for 10 min. The slide was then cooled down to room temperature and the second PET layer was removed from the photoresist. TMMF is a negative type photoresist where the portion of the resist that is exposed to the light becomes insoluble to the developer. Illumination was done under UV light at 500 W for 3.5 s. The mask used was: #405 (array of 6,391 μm by 60 μm square microwells, separated by walls of 10 μm nominal thickness) for In-Tissue device and #407 (22,899 hexagonally arrayed cylindrical microwells of 20 μm diameter with a 20 μm nominal separation between microwells) for the Single Cell device.

Post exposure bake strengthens the cross-linking between the photoresist and the substrate. Post exposure bakes were carried out at 90° C. for 10 min. Development also removes the unilluminated photoresist from the substrate.

In this example of the present disclosure, the development was carried out by the addition of PGMEA developer (incubated for 3 min) followed by the addition of isopropyl alcohol (incubated for 3 min) twice before rinsing it off with DI water. Further details regarding fabrication of this array are shown in Table 1 below.

This example also includes protocols for device fabrication, specifically, as one example, fabrication of the arrays of a double layered microwell.

Due to the uneven surface of the glass filter, having a single layer of TMMF will make the surface of the microwell array chip uneven. This may cause cells to settle down on improperly covered surface between wells rather than in individual wells. Hence, the double layered microwell array was developed.

During the fabrication, certain parameters were changed for the double-layered design when compared to single layered design (discussed above in this Example).

It was experimentally identified that a lamination temperature of 70° C. and a relatively slow roller speed contributes to homogenous adhesion of the TMMF photoresist to the glass filter. The TMMF laminated substrate was subjected to heating at 60° C. for 20 min rather than heating at 90° C. for 10 min so that the structures remained more stable. Lamination carried out at 47° C. with slow speed resulted in the formation of a stable second layer. After the second lamination, the substrate with the photoresist was incubated for 24 hours at 25° C. This prolonged soft bake ensured the stability of the structures after illumination. This process can be continued to add additional layers, such as three, four or more layered structures.

Exposure for 5 s produced structures that were having approximately 70 μm microwell array structures while over-illumination happened at 6 s and 7 s produced microwells with the size of 60-65 μm for the in-tissue device. Conditions for the post exposure bake and development were similar to the single layered design. Analysis of the microwell array images confirms that the size of the microwells is approximately 70 μm and the distance between the two microwells were around 15 μm.

For the double layered and four layered design, the dry film photoresist was laminated twice and four times respectively before illuminating and the conditions at which the photolithography process took place were once again optimized to find the best working conditions. The different process parameters for double layered and four layered microwell array devices are shown in Tables 2 and 3 respectively.

TABLE 1
Photolithography Process-parameters for
single layered micro-well array device

| Process | Process Parameter | Single layer values |
|---|---|---|
| Dehydration bake | Temperature(° C.) | 200 |
| | Holding Time(min) | 10 |
| Lamination | Temperature(° C.) | 90; 70 |
| | Roller Speed(1-9) | 1; 9; 5 |
| Soft Bake | Temperature(° C.) | 100; 95; 60 |
| | Holding time(min) | 10; 20 |
| Exposure | Time(s) | 3.5; 4; 5 |
| Post-exposure Bake | Temperature(° C.) | 90 |
| | Holding Time(min) | 10 |
| Development | PGMEA developer(min) | 3 |
| | Isopropanol(min) | 3 |
| | PGMEA Developer(min) | 3 |
| | Isopropanol(min) | 3 |
| | DI Water(min) | 1 rinse |

TABLE2
Photolithography Process-parameters for
double layered micro-well array device

| | | First layer | Second layer |
|---|---|---|---|
| Process | Process Parameter | values | values |
| Dehydration bake | Temperature(° C.) | 200 | — |
| | Holding Time(min) | 10 | |
| Lamination | Temperature(° C.) | 90; 70 | 47 |
| | Roller Speed(1-9) | 1; 9; 5 | 1 |
| Soft Bake | Temperature(° C.) | 100; 95; 60 | 25 |
| | Holding time(min) | 10; 20 | 24 hours |
| Exposure | Time(s) | — | 4 |
| Post-exposure | Temperature(° C.) | — | 90 |
| Bake | Holding Time(min) | | 10 |
| Development | PGMEA developer(min) | — | 3 |
| | Isopropanol(min) | | 3 |
| | PGMEA Developer(min) | | 3 |
| | Isopropanol(min) | | 3 |
| | DI Water(min) | | 1 rinse |

TABLE3
Photolithography Process-parameters tor four layered micro-well array device

| | | First layer | Second layer | Third layer | Fourth layer |
|---|---|---|---|---|---|
| Process | Process Parameter | values | values | values | values |
| Dehydration | Temperature(° C.) | 200 | — | — | — |
| bake | Holding Time(min) | 10 | | | |
| Lamination | Temperature(° C.) | 70 | 70 | 70 | 70 |
| | Roller Speed(1-9) | 1 | 1 | 1 | 1 |
| Soft Bake | Temperature(° C.) | 60 | 60 | 60 | 25 |
| | Holding time(min) | 20 | 20 | 20 | 1 day |
| Exposure | Time(s) | — | — | — | 8 |
| Post-exposure | Temperature(° C.) | — | — | — | 90 |
| Bake | Holding Time(min) | | | | 10 |
| Development | PGMEA developer(min) | — | — | — | 3 |
| | Isopropanol(min) | | | | 3 |
| | PGMEA Developer(min) | | | | 3 |
| | Isopropanol(min) | | | | 3 |
| | DI Water(min) | | | | 1 rinse |

After fabricating the microwell array device, the surface of the microwell array was silanized by a stamping method using 1% HEPTADECAFLUORO-1,1,2,2-TETRAHYDRODECYL)TRICHLOROSILANE in FC70 (Fluorinated oil). For this purpose, the device and the glass slide were first oxidized with O₂ plasma. 10 μl of the 1% weight % HEPTADECAFLUORO-1,1,2,2-TETRAHYDRODECYL)TRICHLOROSILANE in FC70 was added to the glass slide and heated on a hotplate at a temperature above 100° C. to allow the oil to evaporate (until the shining of the liquid disappears) and then it was stamped to the microwell array device by pressing the glass side to the filter for 1 min.

### Example 5

One use of the microwell array device discussed above is discussed in this Example.

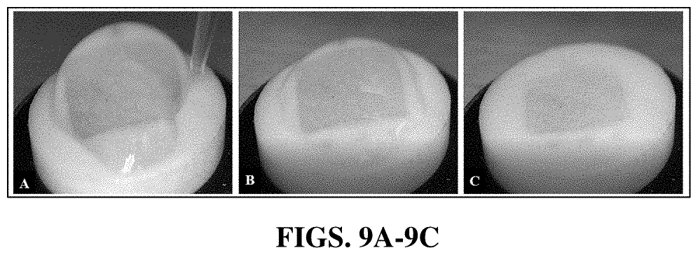

The microwell array device was attached to a filter adapter that was connected to a vacuum unit. Water was added to the device, as seen in FIG. 9A. It was then observed that the water was completely filtered when the vacuum was applied, as shown in FIGS. 9B and 9C. Also, it was observed that the aspiration speed changed according to the incoming vacuum. From these experiments, it was determined that the microwell array chip integrated with the bottom filter does act as a filtration device which can be used to analyze single cells and tissue.

### Example 6

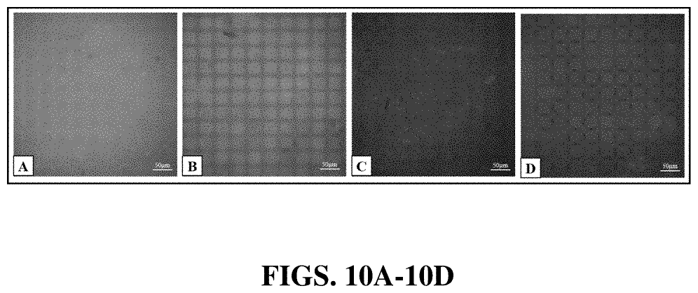

This Example discusses the exchange of a buffer with the microwell array device. Fluorescent micrographs demonstrating the wash and seal-unseal capabilities of the microwell array device are shown in FIGS. 10A-10D. In FIG. 10A, microwells filled with 1 μg/μL fluorescein dye; in FIG. 10B, the microwells were then sealed with an overlay of the FC70 fluorinated oil, trapping the fluorescein dye into individual reactors; in FIG. 10C the microwells are unsealed by aspirating the fluorescein and oil solutions by applying vacuum at the bottom of the device, the microwells were then filled with a solution of 1 μg/μl Rhodamine-2 dye that fully displaced the fluorescein solution; and in FIG. 10D an overlay of FC70 oil was used to re-seal the microwells filled with the Rhodamine-2 dye.

FIGS. 10A and 10C, as well as FIGS. 10B and 10D demonstrate that the microwells after oil sealing can act as independent reservoirs. Based on FIG. 10B and FIG. 10C, it was shown that the designed microwell array device can be used for buffer exchange experiments. This phenomenon when compared to the buffer exchange process performed using the traditional PDMS based microwell array chips is simple, unexpectedly highly efficient and unexpectedly prevents the mixing of the contents in each well.

### Example 7

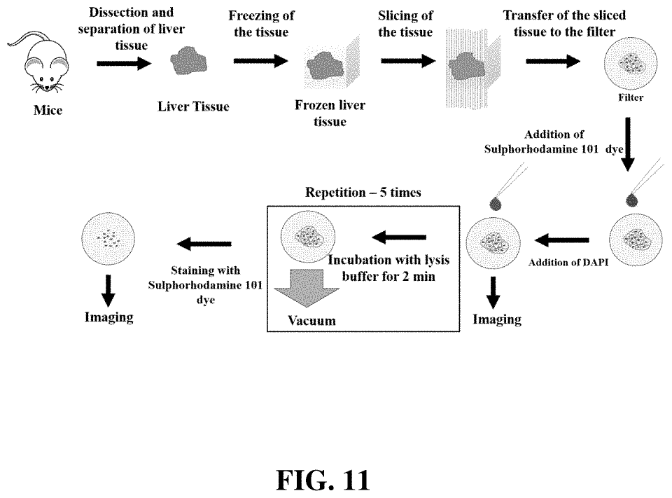

This example discusses the separation and transfer of nuclei from mouse tissues onto a filter, which does not include a microwell array, referring to FIG. 11. FIG. 11 is an illustration of a number of steps for imaging cells taken from the liver of a mouse.

The steps of FIG. 11 include staining the tissue slices with sulphorhodamine 101 to highlight the tissue structure and extracellular material and with DAPI to highlight the position of the nuclei.

Consistent with the steps of FIG. 11, the filter can also keep the nuclei intact when dissolving the tissue without changing the nuclei location. For this purpose, frozen mouse liver tissue samples were sliced using the cryostat and mounted on the glass filter.

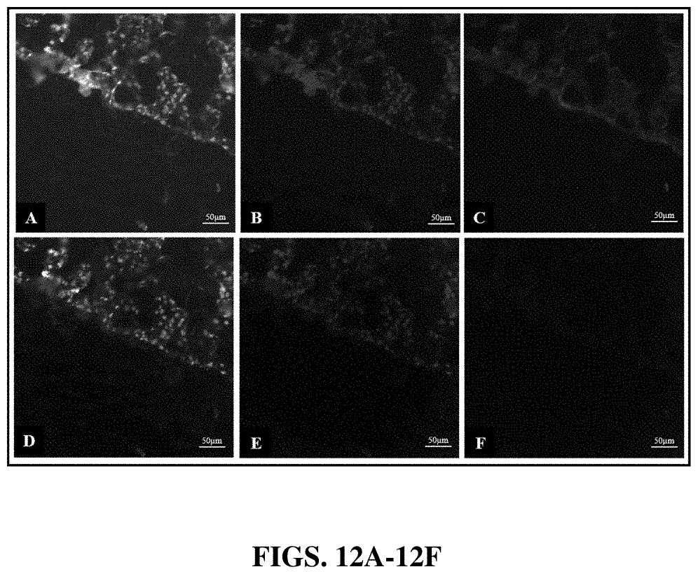

At first the images were taken under brightfield before staining as shown in FIGS. 12A and 12D. These images were taken at the first imaging step, prior to incubation of lysis buffer of FIG. 11. FIGS. 12B and 12C shows the fluorescent micrographs of the tissue and the cell nuclei stained with DAPI and Sulphorhodamine 101 acid chloride before the dissolution of the tissue while FIGS. 12E and 12F shows the fluorescent micrographs after the dissolution of the tissue (imaging step after incubation with lysis buffer of FIG. 11). The tissue was dissolved completely leaving only the nuclei on the filter by a series of process involving chemical treatment with the dissolution buffer followed by aspiration. The dissolution process included 10 repeats.

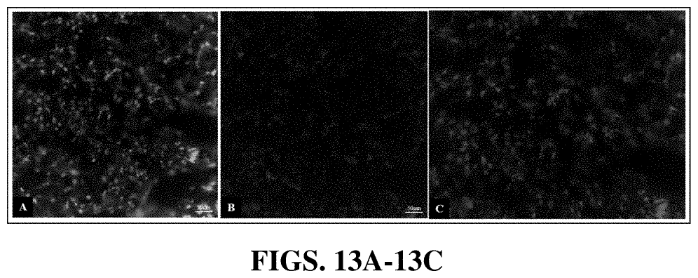

As can be seen in FIGS. 13A-13C fluorescent micrographs of liver tissue are shown. Specifically, in FIG. 13A, fluorescent micrographs of liver tissue nuclei before dissolution, while FIG. 13B is a fluorescent micrograph of the liver tissue nuclei after dissolution. FIG. 13C is a super-imposed image of the nuclei taken before and after dissolution.

To determine the structure and location of nuclei during the dissolution the fluorescent micrographs of the nuclei before and after the dissolution were taken. The hue of the nuclei in the image taken before dissolution was changed to green and then superimposed with the after-dissolution image. After super imposing, the nuclei were tracked manually to track their displacement. The displacement direction specifies whether the nuclei is translocated during the dissolution.

### Example 8

This example is directed to a protocol for fabricating microwell arrays on a glass filter disk, using Ordyl® SY-320, which is an aqueous solvent type dry film photoresist for micro-electromechanical systems (MEMS) applications.

The following Materials were used in this example: Ace Glass 15 mm Diameter Filter Disks with Porosity (E) 4-8 um pores; Ordyl® SY320, stored in the dart at about 4 C; Ordyl® Developer; 1% TCS+HFE7500; Armor Etching Cream; Argon Gas; Dry Nitrogen Gas; Isopropanol Alcohol 99%; 2% Hellmanex+DI Water; DI Water; 250 mL beaker, 2×50 mL beakers; Disposable Pipettes; 4× Scintillation Vials; Small Metal Spatula; Metal Tongs; Aluminum Disk Holder Plate; Magic Scotch Tape; and a Scalpel. In conjunction with these materials, A sonicator, hot plates, a laminator and UV illumination stages were used.

In this example, the first step is preparation of the glass filter disk, the method to prepare the glass filter disk are as follows:

#1 Pre-washing and Drying of Ace Glass 15 mm Diameter Filter Disks with Porosity (E) 4-8 um pores, which includes the step of: cleaning glass filters with 2% Hellmanex Solution+DI water with Sonication for 15 min; If the solution is clear after sonication is over, move to next step; If solution is foggy, clean glass filters again in the same manner with a newly prepared solution (Always use a new solution).

#2 currently using 250 mL Beaker, 4 mL of Hellmanex, fill to 200 mL with DI water. After sonication is complete, place each individual filter into a scintillation vial without bottle cap and heat on a hotplate at 120 degrees C. for 30 min to 60 min until it is virtually dry (these bottles should be saved and reused).

#3 Surface Treatment with TCS+HFE 7500 1% Solution. Prepare solution by measuring 2 grams of HFE 7500 first, then adding 10 ul of TCS (Tetrachlorosilane) [green cap bottle HEPTADECAFLUORO-1,1,2,2-TETRAHYDRODECYLTRICHLOROSILANE—FDTS; Product Code: SIH5841.0]. When adding TCS, turn on argon gas from tank, set pipette to proper volume and lock, have the vial with HFE 7500 open, open the TCS bottle under argon gas flow and keep it under the gas the entire time the bottle is open to displace the moist air, take pipette and press on a tip with the other hand, draw out 10 ul and inject into the HFE, then discard the tip, and fill both the cap and TCS bottle with argon before closing it, wrap bottle with parafilm and return the bottle to dessicator. Then, place the dried filter disk into the solution, if the filter is not submerged, when removing the filter out, mark the surface facing up with an X, 60 min submersion. After 1 hr, return the filter onto 120 degree C. hotplate for 30 min to evaporate the HFE 7500 from the filter.

#4 Always open TCS in the presence of Argon gas, the gas is heavier than air and will displace the humid air that will crystallize the liquid TCS making it unusable, with anything that comes into contact with TCS, rinse under water before discarding, TCS is extremely harmful if inhaled.

#5 Surface Etching with Armor Etching Cream. Using a swab or pipette, scoop Armor Etching Cream onto the surface that had been properly submerged in the 1% TCS solution, ensure the entire surface is covered (no uncovered edges around the circumference of the disk), leave for 5 min. After 5 min, remove and rinse in DI water by taking the filter with tongs and holding it so that the surface that was etched is facing the flow of water, keep filter close to the bottom of the sink to maximize pressure of water to remove etching cream.

#6 Final Washing. Prepare 2% Hellmanex solution with DI water and submerge the filter etch side facing up, leave to sonicate for 15 min. After sonication is complete, bring to the clean room to be rinsed thoroughly with DI water and then with IPA. Dry with dry nitrogen gas.

#7 Dehydration Baking. Bake filter at 200 degrees C. for 10 min to assist with lamination process, always have the etched side facing up.

In this example, the second step is patterning using double layer Ordyl® SY320 as follows:

#1 Ordyl® Lamination, in the absence of white and UV light. Turn on and set laminator to 110 degrees C. with speed 4 (1.15 m/min); Clean aluminum plate with IPA and dry with nitrogen; Cut Ordyl® sheet into a square that fits over the entire disk surface (filter is 15 mm in diameter, so a 15 mm×15 mm square can be used); Take filter from dehydration and place into aluminum lamination plate; Peel 1st plastic cover film away from the Ordyl® material, place down onto the filter and tap with pressure one single corner, the corner that is pressed will be pushed first into the laminator, laminate 2×; Using a scalpel, trace between the filter perimeter and the aluminum plate hole to extract the filter from the plate; Leave for 30 min to cool down to room temperature, the Ordyl® material should return to room temperature, leave in a dark cool place; Peel second plastic cover film only if the Ordyl® material is room temperature, peel using one swift motion; take a second piece of Ordyl® material and repeat the lamination process with the second layer of Ordyl® material; Let cool for 30 min until room temperature; and Laminate again at room temperature (18-20 degrees C.) to improve Adhesion of the Ordyl® material to filter (optional).

#2 Illumination with UV light at 500 W. Place filter with laminated double layer of Ordyl® material onto stage; Peel the secondary plastic layer from the Ordyl® material surface using one motion to release the film from the surface, this will prevent any stray marks on the Ordyl® material surface; place mask over the filter, use your thumb to gently press down until interference patterns can be seen between the Ordyl® material surface and the mask; Illuminate under 500 W UV light for 4 s; Post Exposure Bake at 150 degrees C. with the Ordyl® material's surface of the filter facing up on top of a clean glass slide for 10 min.

#3 Develop with Ordyl® Developer by hand. Using a pipette, rinse the Ordyl® material's surface in a small glass beaker, tilt the beaker so that the filter is not submerged in the solvent, continuously use the pipette to rinse evenly over the surface in a zigzag pattern, do so for 3.5 min. then in a separate container rinse with IPA, with tongs hold the filter and with the same motions, rinse over the surface with the IPA bottle, pour away the waste then submerge in IPA and swirl the beaker thoroughly ×3 (approximately 2 min); dry with nitrogen gas; Hard Bake @85° C. for 10 min.

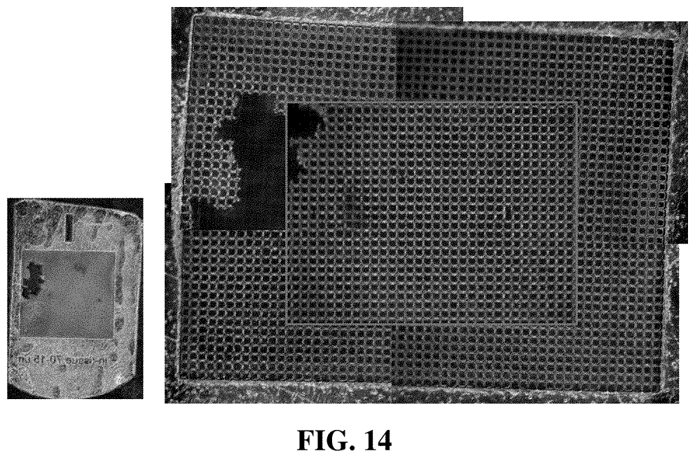

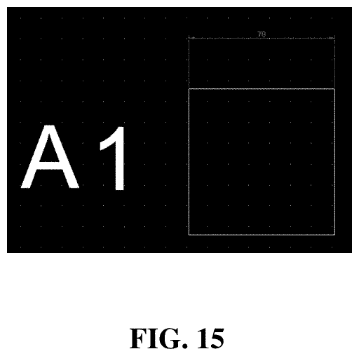

This step of the method results in a fabricated microwell array, as shown in FIG. 14, which is an image of the array obtained with a stereomicroscope. As can be seen from FIG. 14, the left image is an image of the fabricated microwell array, while the right image is a magnified view thereof. As can be seen more clearly in the right image, a plurality of wells are formed.

### Example 9

This example is directed to a protocol for preparing a microwell array on a 0.2 μm syringe membrane filter membrane.

An aim of this protocol is to create an array that can be used to collect bacteria and run polymerase chain reaction (PCR) on. A past problem has been the difficulty to collect and run tests on compartmentalized samples, however, polystyrene is a fundamentally biocompatible material for cell culture work. This provides the advantage of simultaneously collecting then detecting of bacteria for the study of, for example, Methicillin-resistant Staphylococcus aureus (MRSA).

Another aim is to have a single sided through-hole array of microwells with high resolution in polystyrene that are of a standardized dimension. The microarray filter will be intended for collecting bacterial cells into bins that can be easily labeled and distinguished after collection and upon inspecting and diagnostics. The polystyrene microwell array layer can be bonded to a 0.2 μm pore size syringe filter membrane. The combined device will have several purposes, one of which is a 0.2 μm membrane to filter out bacteria from a suspended solution.

Another aim is to successfully bond imprinted polystyrene array of microwells onto 0.2 μm membrane filters (Polytetrafluoroethylene (PTFE) and Cellulose Acetate).

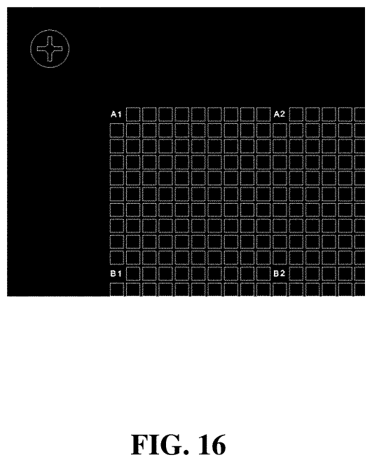

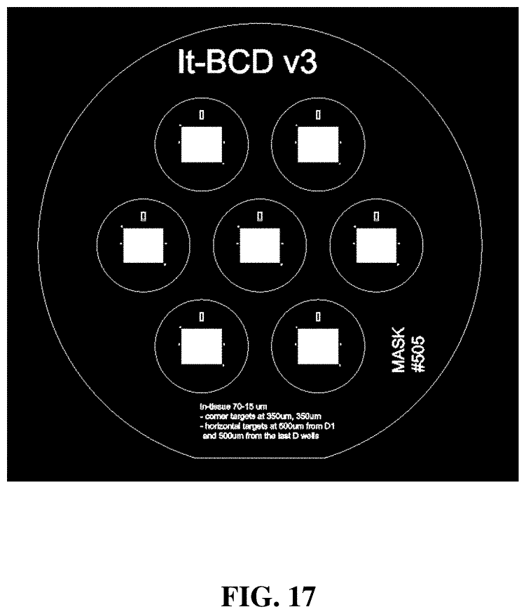

FIG. 15 is a design of one microwell having a dimension of 70 μm by 70 μm, which can be distanced apart 15 μm from each other in an array. Such an array is shown in FIG. 16, which illustrates a portion of a microwell array, specifically rows 1-8 and columns A-G of an array. FIG. 17 is a design that can be used to create a photomask to form a number of microwell arrays by photolithography.

In this example, the following definitions are relied on: “microwell Arrays” are an arrangement of wells with a volume in nanoliters well suited for confining cells: “photolithography” is a process by which micro patterns can be constructed on the surface of a substrate by using light (UV) to transfer the geometric design from a photomask to a master, including a photosensitive chemical to produce a master when a dry film photoresist such as TMMF or Ordyl® is used as the substrate: “master” is the substrate with the desired pattern mold used for casting, the prime master is made with a silicon wafer; “soft lithography” is the fabrication process of casting the master with an elastomer such as PDMS (polydimethylsiloxane) where the geometry of the relief of the master form the negative 3D spaces which can be channels, wells, etc.; “intermediate PDMS slab” refers to the negative of the silicon wafer master mold that is used to cast a master in 5:1 PDMS; and “imprinting stack” is the stacked order of materials positioned under pressure before thermal exposure, the order and placement of these materials allows for the effective transfer of the microwell array design onto the polystyrene film.

This example used the following materials: a Silicon Wafer Master —utilizing photolithography, produce the microwell array pattern on a silicon wafer (positive design); Acrylic Jigs and Hardware—Custom laser cut housing for wafer master to cast first negative PDMS slab; Sylgard™ 184 PDMS—Polymer for intermediate negative of design; Goodfellow 30 μm Polystyrene Films—Thermoplastic for imprinting microwell array; 0.2 μm Syringe Filter Membranes—filter membranes; Metal Tongs—Safety to remove heated elements; Autoclave gloves—Safety for removing heated elements; Needle nose forceps—for manipulation and removal of polystyrene microwells; Aluminum Plates—for containment, should the plastic flow out and over the PDMS slabs; Large Binder Clamps; Tape; 15 cm Diameter Petri Dishes; High Vacuum Grease: Medium Mixing Boats; Goggles; and Angle Nose Slip Joint Pliers. Along with these materials, a desiccator, scale, bandsaw, hot press, 80° C. oven, and an additional oven reserved for temperature changes.

In this example, the first step is photolithography, the photolithography method is as follows: perform photolithography for about 2 hours to create a positive master onto a silicon wafer (in this example, negative photoresist SU-8-2050 was used, along with sheet 044 Mask #505, with a desired height of 50-55 μm) followed by TCS treatment overnight.

In this example, the second step is soft lithography for about 3.5 to about 5 hours for creating a PDMS slab followed by TCS treatment overnight. To effect this step, steps A-C are followed. Step A is using an Intermediate Negative PDMS Slab, which will be used as the mold to cast a new master using 5:1 Sylgard™ 184 PDMS. Step A includes Mixing 17 g of 10:1 (Part B:A) of Sylgard™ 184 PDMS in a medium white boat and de-gas using dessicator for 5 min, turning on the vacuum and releasing once vacuum is set (cycling the vacuum and release will speed up de-bubbling); securing Silicon Master in an acrylic jig, the acrylic top frames come in a variety of thicknesses and framing shapes, choose one accordingly for the boundary shape of the pattern; top frame of 1.7 mm or 2.5 mm thickness to produce PDMS slab thickness of 2.33 mm or 3.53 mm (this securing step includes (placing the wafer on the square acrylic base; utilizing a spacer to keep the master centered and to prevent the top acrylic frame from placing too much pressure on the wafer; placing top acrylic frame over the wafer and spacer; using Hex screws spaced by 2 washers, screw securely the top acrylic frame to the base; and observe that the wafer is centered within the spacer by raising it over a light to clearly see the outline between the wafer and the spacer); pouring de-gassed PDMS onto the pattern until there is a doming effect in the way the PDMS rests within the frame volume, first pour always compensates to fill in small spaces between the pieces of the jig and the wafer; setting on a leveled surface in the 80 C oven and leave for minimum 3 hrs (max overnight); once cured, cut out and tape with Magic Scotch Tape; and by vapor deposition, TCS treat the slab overnight. Step B is, on the next day, Positive PDMS Master Fabrication with 5:1 (B:A) Ratio of Sylgard™ 184 (6 hrs+overnight), which includes mixing 30 g of 5:1 of Sylgard™ PDMS for a stiffer polymer, de-gas per usual; with the Intermediate Negative PDMS slab with pattern side facing up, place into a petri dish, ensure that the slab adheres to the surface of a petri dish by seeing that there are no air bubbles trapped between the PDMS and petri dish when placing it down, alternatively if there is concern the PDMS slab will not adhere, take high vacuum grease and with a gloved finger, spread a thin even coating on the smooth PDMS side to adhere it more firmly; pouring de-gassed 5:1 PDMS in and let it bake in 80° C. oven for 5 hrs to ensure the PDMS has entirely crosslinked (this timing is important and impacts the stiffness, shorter and it may be too flimsy/flexible, the longer it remains in the oven the stiffer the polymer and it may crumble); removal from oven, peel the combined slab from petri dish, carefully remove one slab from the other by cutting away the edges and gently peeling them apart, and TCS treated overnight. Step C is Blank PDMS Slabs (3.5 hrs+overnight), which includes mixing 10:1 Sylgard 184 and pour into a petri dish (mass is dependent on the diameter of the dish, a slab thickness of 3 mm is ideal); letting cure in the 80° C. oven for 3 hrs; once cured, tape and remove from the petri dish, cut into squares 1×1 cm²; and TCS Treat with Positive 5:1 PDMS Master overnight.

In this example, the third step is imprinting with TCE treated 5:1 PDMS master. In this step initial testing was conducted. To imprint the 30 μm polystyrene films, a temperature of 110° Celsius for a duration of 3 days clamped in the oven was used. Simultaneously, temperatures of 130° Celsius and 160° Celsius were used to determine if higher temperatures would allow for faster imprinting. The following process was used to create double and single imprinted microwell arrays, as shown in reference to the figures.

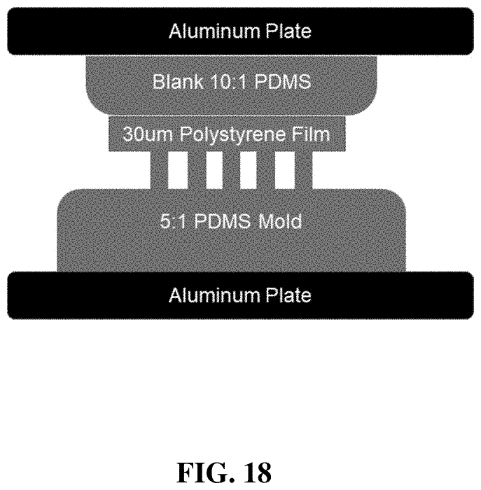

FIG. 18 is an illustration of an example of the Stacking Order of imprinting elements for single sided imprints; Base-aluminum, 5:1 PDMS Master, polystyrene (PS) 30 μm film, blank PDMS slab, aluminum-top. For double-sided imprinted, 2 5:1 PDMS Master Molds were used and turned 30 degrees with the 30 μm film sandwiched in the middle. When using binder clamps in the oven, 4 additional aluminum plates were used when under binder clamps to maintain enough pressure on the imprinting stack.

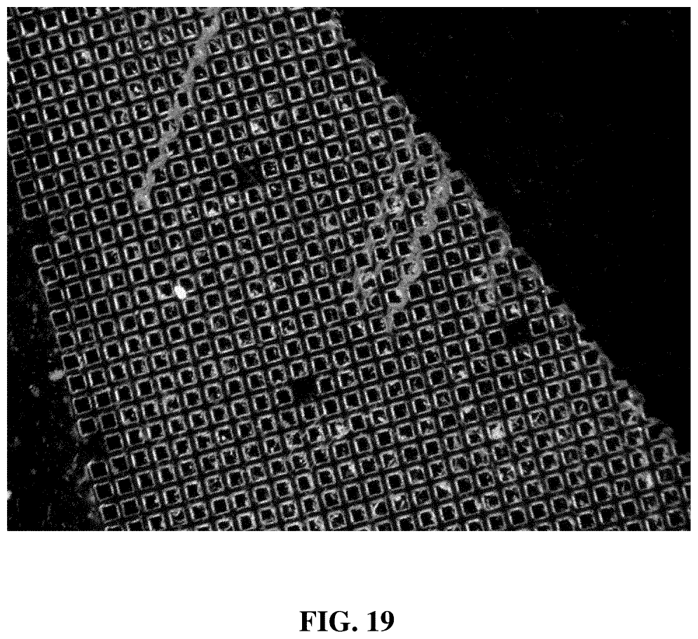

FIG. 19 is an Image of single-sided microwell array, this particular imprint was compressed during cooling leaving it wrinkle-free.

Another example of bonding to a 25 mm syringe membrane at 0.2 μm pore size (Cellulose acetate, PTFE) is discussed in view of the following steps.

After 10 min imprinting is complete, turn off the hot press and wait for 5 min, then release pressure slowly so that the PS film does not get accidentally peeled or shifted, followed by removing the whole stack using autoclave gloves and tongs if necessary, and once removed, turn the machine on again to return to 160° C., then with the stack placed onto the bench, remove the top aluminum plate and peel the blank PDMS slab away carefully without removing the film from the master, followed by using a tabletop bandsaw with proper plastic saw blade ring, cut open 25 mm syringe by handling with angle nose slip joint pliers and cutting away the circumference in straight lines, then once the edges have been cut away, using another set of pliers or by hand, pry away the housing to reveal the thin membrane, then place cellulose acetate or PTFE membrane filter with smooth side down onto imprinted PS, then replace blank slab and aluminum top plate, followed by placing back onto hot press with the same pressure to prevent slipping of stack for 3 min, then turn off, and leave to cool on the hot press for 30 min, after 30 min, Remove the still, very, hot stack carefully from the hot press and place down on the bench to cool, after cooling, lift away the top plate with tongs and autoclave gloves, then the blank slab with tongs grasping and tilting to peel away slowly from the cellulose acetate, using tongs, remove the PDMS master+PS+Cellulose acetate from the base aluminum plate and set onto glass slide, with pointed tweezers, carefully release the PS Microwell and membrane filter and place it between an inverted petri dish top and bottom with magnets gently snapped on either side, and finally let cool completely, the magnets and flat surface prevent wrinkling/curling (30 min).

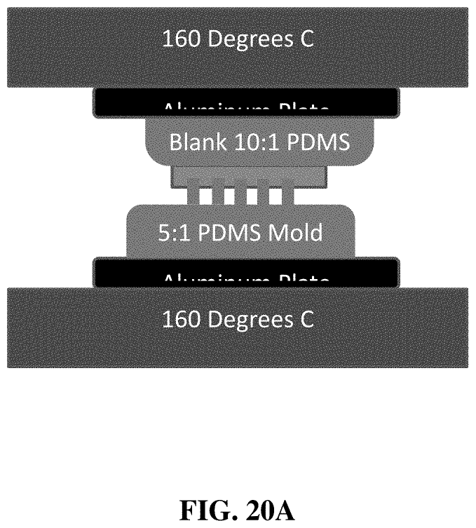

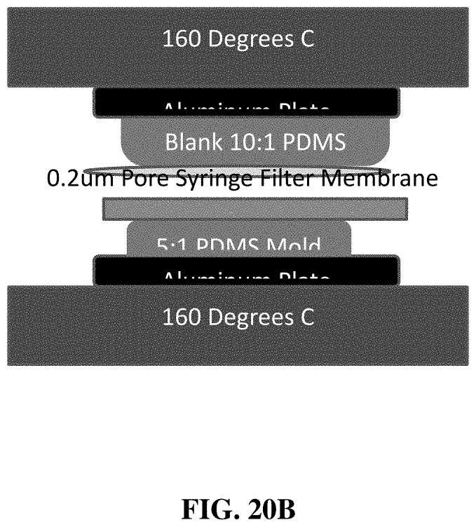

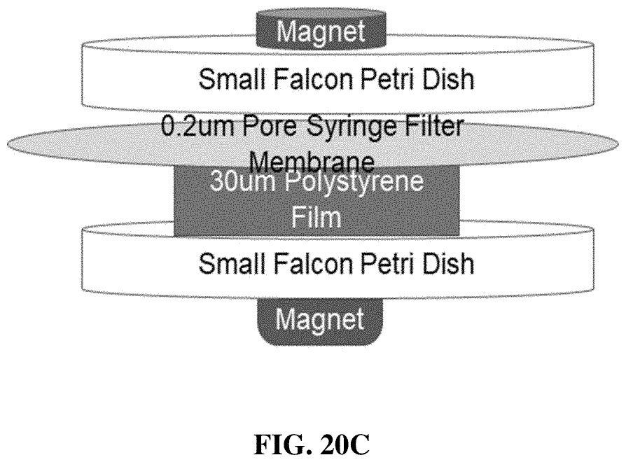

FIGS. 20A-20C is an illustration of the different steps used in the above example. FIG. 20A illustrates the first step of imprinting the thermoplastic film. FIG. 20B illustrates the second step of pressing a thin membrane with the thermoplastic imprinting assembly. FIG. 20C illustrates the third step of pressing the thin membrane during cooling using a magnetic clamping method. This, FIG. 20A-20C illustrate a compression method to reduce/prevent deformations and wrinkling during cool down using inverted petri dishes placed on either side of the filter device and pressed with magnets. The thin membrane can be composed of cellulose acetate, PTF and/or polycarbonate, for example, and can include any suitable pore size, such as about 0.2 microns to about 5 microns.

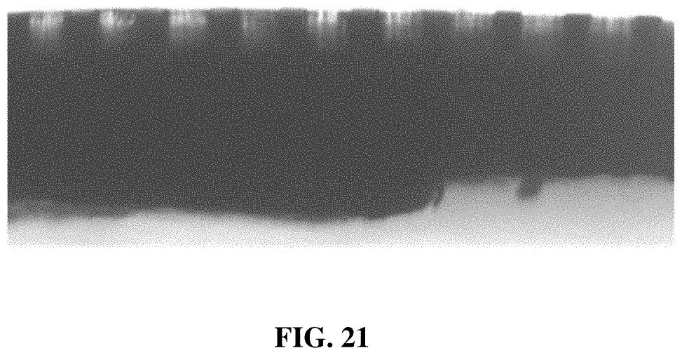

FIG. 21 is a photograph, taken with a Nikon Diaphot at 10× magnification, showing a single sided through-hold imprinted 30 μm thick polystyrene film bonded to cellulose acetate 0.2 μm pore syringe membrane.

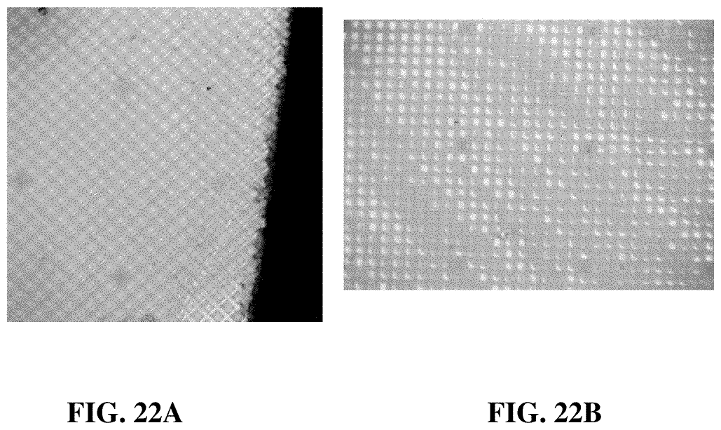

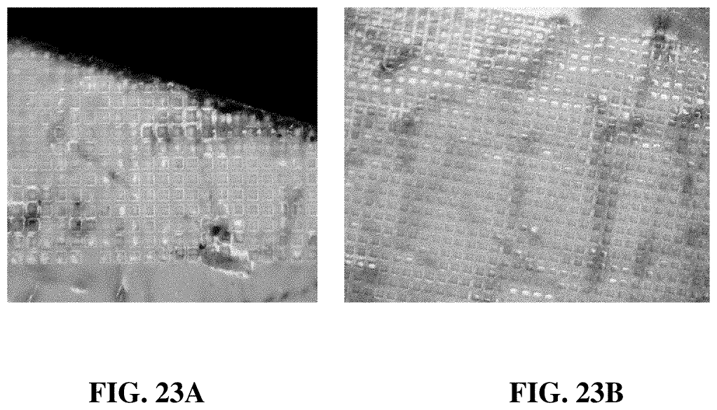

FIGS. 22A and 22B are images of a single sided polystyrene filter bonded to 0.2 μm cellulose acetate syringe filter membranes, formed according to the method discussed above. FIGS. 23A and 23B are images of a single sided polystyrene filters bonded to 0.2 μm PTFE syringe filter membranes, formed according to the method discussed above.

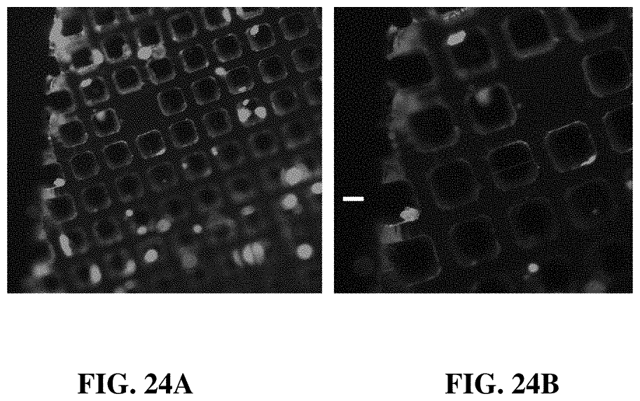

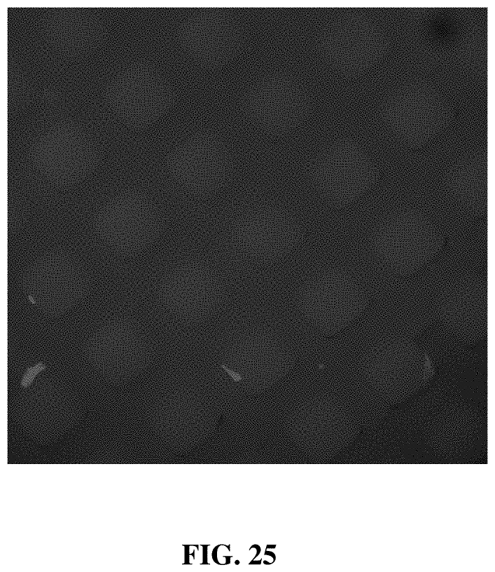

FIGS. 24A and 24B are Nikon AZ100 images of a single sided polystyrene filter bonded to 0.2 μm PTFE syringe filter membranes, formed according to the method discussed above. The images were captured at DAPI 64× gain, for FIG. 24A, the auto exposure was 200 ms and 4× magnification, for FIG. 24B, the auto exposure was 500 ms and 7× magnification.

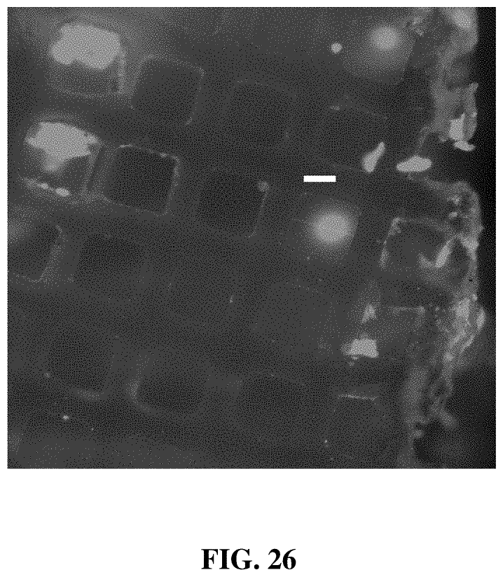

FIG. 25 is an image of a PS microwell array on a cellulose acetate membrane of 0.2 μm pore size, taken with a DAPI filter set 30 ms exposure, 64× gain, 7× magnification with a Nikon AZ100M scope. FIG. 26 is an image of a PS microwell array on a PTFE membrane of 0.2 μm pore size, taken with a DAPI filter set 150 ms exposure, 64× gain, 7× magnification with a Nikon AZ100M scope.

In this example, several working variables were determined as better than others, some of which include the following. A PDMS Master with a 5:1 ratio works best under pressure, the master should be a thin slab. When imprinting for through-holes, the master needs to be placed first on the base for plastic to flow away, being drawn away and around the raised pattern of the PDMS. Imprinting works best at 160° C., but can work at temperatures between 130-180° C., heating time must be prolonged. A stiff but flexible material should be used to push the PS over the PDMS Master, in this case another PDMS master or a blank PDMS slab. To reduce/prevent wrinkling of the PS filter once imprinted, the film should remain compressed until it is cooled to room temperature. For films thicker than 30 μm, there should be an adjustment to the master thickness. For films thicker than 30 μm, temperature and pressure can be kept constant, but duration must be prolonged; trial and error testing of pressed falcon petri dish chips (1 cm×1 cm) of thicknesses 150-200 μm required imprinting times of 3 hrs.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A microwell array comprising a plurality of wells, wherein at least one well of the plurality of wells comprises:
a glass bottom surface configured to receive a vacuum pressure and configured to transmit the vacuum pressure to an interior volume of the well, wherein the interior volume of the well is comprised of a side wall and the bottom surface; and
the side wall is hydrophilic, wherein the glass bottom surface is either hydrophobic or hydrophilic.

2. The microwell array of claim 1, wherein the plurality of wells are etched into a porous glass.

3. The microwell array of claim 2, wherein the bottom surface comprises the porous glass.

4. The microwell array of claim 1, wherein the side wall is non-porous.

5. The microwell array of claim 1, wherein the side wall is porous.

6. The microwell array of claim 1, wherein the plurality of wells are deposited on an upper surface of the glass bottom surface.

7. The microwell array of claim 6, wherein the side wall comprises a deposited metal.

8. The microwell array of claim 1, wherein portions of the side wall of adjacent wells are hydrophobic.

9. The microwell array of claim 1, wherein the glass bottom surface has a pore size of about 4 microns to about 8 microns.

10. The microwell array of claim 1, wherein the glass bottom surface has a pore size of about 2 microns to about 2.5 microns.

11. The microwell array of claim 1, wherein the glass bottom surface has a pore size of about 0.9 microns to about 1.4 microns.

12. The microwell array of claim 1, wherein the glass bottom surface has a pore size of about 10 microns to about 20 microns.

13. The microwell array of claim 1, further comprising a plurality of beads in each of the plurality of wells, wherein the plurality of beads are configured to fill a plurality of pores in the glass bottom surface.

14. The microwell array of claim 1, wherein at least one of the glass bottom surface and the upper edge are fluorophilic.

15. The microwell array of claim 1, further comprising a plurality of first barcodes, wherein the plurality of first barcodes are printed on at least one of the side wall and the bottom surface.

16. The microwell array of claim 15, further comprising a plurality of second barcodes, wherein the plurality of second barcodes are printed on at least one of the side wall and the bottom surface.

17. The microwell array of claim 15, wherein the plurality of first barcodes are attached to a first particle.

18. The microwell array of claim 17, wherein the plurality of first barcodes are attached by grafting, printing or synthesizing of the first barcodes directly onto a surface of the first particle.

19. The microwell array of claim 17, wherein the interior volume of the well comprises the first particle.

20. The microwell array of claim 16, wherein the plurality of first barcodes are attached to a second particle.

21. The microwell array of claim 20, wherein the plurality of first barcodes are attached by grafting, printing or synthesizing of the first barcodes directly onto a surface of the second particle.

22. The microwell array of claim 20, wherein the interior volume of the well comprises the second particle.

23. The microwell array of claim 1, wherein a space between the upper edge of two adjacent wells of the plurality of wells forms an upper surface, wherein the upper surface is hydrophobic.

24. The microwell array of claim 1, wherein the at least one well of the plurality of wells further comprises an upper edge of the side wall, wherein the upper edge of the side wall is hydrophobic.

25. A method of cell analysis, the method comprising:
add one or more cells to one of a plurality of wells of a microwell array etched into a porous glass;
apply a vacuum to a bottom surface to one well of the plurality of wells;
add one or more buffers and/or reagents to the one well; and
apply a sealing oil over a top surface of the one well.

26. The method of claim 25, further comprising performing a polymerase chain reaction (PCR) to the one or more cells after the step of applying the sealing oil.

27. The method of claim 25, further comprising removing the sealing oil and removing the one or more buffers and/or reagents from the one well.

28. The method of claim 27, wherein the one or more buffers and/or reagents comprise a lysing agent.

29. The method of claim 25, wherein the one well further comprises at least one of magnetic beads and grafted molecules.

30. A method of forming a microwell array, the method comprising:
covering a glass filter in a silanizing oil;
evaporating at least a portion of the silanizing oil from the glass filter;
covering at least a portion of a surface of the glass filter with an etching material;
removal of the etching material after a period of time;
sonicating the glass filter;
heating the glass filter;
applying a photoresist to the glass filter;
heating the photoresist and the glass filter; and
illuminating the photoresist.

31. The method of claim 30, further comprising before the illuminating step,
applying a second photoresist to the glass filter;
heating the second photoresist and the glass filter; and
illuminating the second photoresist and the glass filter.

32. The method of claim 30, further comprising performing a polymerase chain reaction (PCR) to the one or more cells after the step of applying the sealing oil.

33. The method of claim 30, further comprising removing the sealing oil and removing the one or more buffers and/or reagents from the one well.

34. The method of claim 33, wherein the one or more buffers and/or reagents comprise a lysing agent.

35. The method of claim 30, wherein the one well further comprises at least one of magnetic beads and grafted molecules.

36. A method of forming a microwell array, the method comprising:
applying a pressure to a positive silicon master and a silicon slab, wherein a polystyrene film is between the positive silicon master and the silicon slab; and
heating the positive silicon master, the silicon slab and the thermoplastic film for a period of time; and
adding a thin porous membrane between the thermoplastic and the silicon slab.

37. The microwell array of claim 36, wherein the thin porous membrane is a cellulose acetate membrane filter that has a pore size of about 0.2 microns to about 5 microns.

38. The microwell array of claim 36, wherein the thin porous membrane is a PTFE membrane filter that has a pore size of about 0.2 microns to about 5 microns.

39. The microwell array of claim 36, wherein the thin porous membrane is a polycarbonate membrane filter that has a pore size of about 0.2 microns to about 5 microns.

40. The method of claim 36, further comprising performing a polymerase chain reaction (PCR) to the one or more cells after the step of applying the sealing oil.

41. The method of claim 36, further comprising removing the sealing oil and removing the one or more buffers and/or reagents from the one well.

42. The method of claim 41, wherein the one or more buffers and/or reagents comprise a lysing agent.

43. The method of claim 36, wherein the one well further comprises at least one of magnetic beads and grafted molecules.
